(19) United States
(12) Patent Application Publication
(10) Pub. No.: US 2006/0247512 A1
(43) Pub. Date: Nov. 2, 2006

(54) Patient supported in-bore monitor for MRI

(75) Inventors: Robert A. Harwell, Orlando, FL (US); Arthur R. Weeks JR., Oviedo, FL (US); Stephen Douglas Fisher, Winter Springs, FL (US); Jorgen Kilden-Pedersen, Orlando, FL (US)

Correspondence Address: QUARLES & BRADY LLP, 411 E. WISCONSIN AVENUE, SUITE 2040, MILWAUKEE, WI, 53202-4497, US

(73) Assignee: Invivo Corporation

(21) Appl. No.: 11/080,743

(22) Filed: Mar. 15, 2005

(51) Int. Cl. A61B 5/05

(52) U.S. Cl. 600/410

(57) ABSTRACT

A portable, wireless patient monitor may be placed with the patient in the bore of an MRI machine eliminating the need for separate cabling between the MRI machine and an external monitoring unit. In one embodiment, the patient monitor may be attached to the patient's shoulder by a harness or the like which may also serve to corral leads between the patient monitor and the patient.

### CROSS-REFERENCE TO RELATED APPLICATIONS

This application is a continuation-in-part of U.S. Application ______ filed ______.

### STATEMENT REGARDING FEDERALLY SPONSORED RESEARCH OR DEVELOPMENT

### BACKGROUND OF THE INVENTION

The present invention relates generally to electronic patient monitors, and in particular, to a wireless patient monitor suitable for use in the severe electromagnetic environment of a magnetic resonance imaging machine.

Magnetic resonance imaging (MRI) allows images to be created of soft tissue from faint electrical resonance signals (NMR signals) emitted by nuclei of the tissue. The resonance signals are generated when the tissue is subjected to a strong magnetic field and excited by a radio frequency pulse.

The quality of the MRI image is in part dependent on the quality of the magnetic field which must be strong and extremely homogenous. Ferromagnetic materials are normally excluded from the MRI environment to prevent unwanted forces of magnetic attraction on these materials and distortion of the homogenous field by these materials.

A patient undergoing an MRI “scan” may be received into a relatively narrow bore, or cavity in the MRI magnet. During this time, the patient may be remotely monitored to determine, for example, heartbeat, respiration, temperature, and blood oxygen. A typical remote monitoring system provides “in-bore” sensors on the patient connected by electrical or optical cables to a monitoring unit outside of the bore.

Long runs of cables can be a problem because they are cumbersome and can interfere with access to the patient and free movement of personnel about the magnet itself.

### BRIEF SUMMARY OF THE INVENTION

The present invention provides a wireless patient monitor that may be placed in the bore of the MRI machine with the patient during scanning. Resistance to the extreme electrical environment within the bore is provided by a shielding system that works with the wireless transmitter in the patient monitor. The shielding system is also designed to minimize eddy current induced vibration allowing the patient monitor to be attached to the patient. In this latter case, the patient monitor can be attached to the patient's shoulder to provide good access to data sent wirelessly from the patient monitor to a remote receiver. Wireless communication eliminates the cabling which must pass from the bore to remote monitoring equipment and the ability to place the monitor in the bore itself reduces the length of leads communicating with sensor elements on the patient, for example, electrodes or SPO₂ optics.

Specifically then, the present invention provides an electronic patient monitor providing at least one sensor for receiving a patient signal from the patient and having a transmitter system for transmitting data communicating the patient signal. A shield housing surrounds the electronic patient monitor to block free space radio frequency signals therethrough allowing operation of the electronic patient monitor within a bore of the MRI machine during scanning and to suppress eddy currents from the MRI gradients, reducing vibration of the monitor. An antenna attaches to the outside of the shield housing and communicates with the wireless transmitter through an aperture in the shield housing.

Thus it is one object of at least one embodiment of the invention to provide a monitor unit that may be near to or on the patient during scanning without excessive vibration.

The sensor system may include a shell surrounding the shield housing.

It is thus another object of at least one embodiment of the invention to provide a housing that may be safely placed on or near the patient and that is resistant to damage.

The antenna may be covered by the shell and may be, for example, a micro strip antenna.

It is thus another object of at least one embodiment of the invention to prevent the antenna from interfering with placement of the monitor.

The shield housing may comprise separate sections joined by eddy current blocking capacitors.

It is thus another object of at least one embodiment of the invention to provide a patient monitor that may be comfortably placed on the patient without eddy current induced vibration as might be disturbing or uncomfortable to a patient touching the monitor.

The shield housing may be a substantially rectangular parallelepiped having each face electrically joined to an adjacent face by DC blocking capacitors.

It is thus another object of at least one embodiment of the invention to provide a manufacturable shield housing that provides for ample contained volume.

The shield housing may be mesh.

It is thus another object of at least one embodiment of the invention to provide a lightweight shield material that accommodates the viewing of a display that may be associated with the monitor.

The monitor may include a display, for example, an LCD panel.

It is thus another object of at least one embodiment of the invention to provide an in-bore patient monitor that can also serve as a primary patient monitor outside of the MRI room.

The electronic patient monitor may include an LED visible outside the shield housing through at least one aperture in the shield housing.

It is thus another object of at least one embodiment of the invention to provide a display that allows verifying the operation of the patient monitor from outside the bore of the magnet simply by inspection.

The system may include a mount adapted to hold the electronic patient monitor to the patient.

It is thus another object of at least one embodiment of the invention to reduce possible stress on the leads attached to the patient by attaching the patient monitor to the patient.

The mount is adapted to hold the electronic patient monitor with the antenna removed from the patient, and when an LED is used, to allow the LED to be visible by a person observing the patient outside the bore of the magnet. Preferably this mount is to the patient's shoulder.

It is thus another object of at least one embodiment of the invention to allow a location of the patient monitor to improve communication between the patient monitor and remote sensing systems.

The patient mount may include a harness fitting around the patient's shoulder.

It is thus another object of at least one embodiment of the invention to provide a convenient means of attaching the patient monitor to the patient.

The mount may include a harness supporting leads attaching the sensors to the patient.

It is thus another object of at least one embodiment of the invention to provide a method of managing the leads between the sensor and the patient to prevent them from being tangled or obstructing access to the patient.

The sensor may include batteries held within the shield housing to power the electronic patient monitor.

It is thus another object of at least one embodiment of the invention to provide a source of portable power that is compatible with operation in the MRI machine during scanning and that eliminates the need for remote power sources.

These particular objects and advantages may apply to only some embodiments falling within the claims and thus do not define the scope of the invention.

### BRIEF DESCRIPTION OF THE DRAWINGS

FIG. 1 is a simplified, perspective view of an MRI system showing the MRI magnet and the location of an in-bore patient unit and an out-of-bore receiving unit;

FIG. 2 is a block diagram of the patient unit of FIG. 1 configured for ECG collection and showing blocks of a microprocessor-controlled diversity transmitter employing a contained strip antenna and an on-board display;

FIG. 3 is a block diagram of the receiving unit of FIG. 1 showing multiple diversity receivers with switched antennas communicating with a programmable controller to select accurate data for outputting to a display screen;

FIG. 4 is a timing diagram of digital data packet transmitted using the diversity system of the present invention with one packet enlarged showing time diversity transmission of ECG data with a trailing error-correction code;

FIG. 5 is a figure similar to that of FIG. 4 showing a digital data packet that may be transmitted from the processing unit to the in-bore patient unit for providing commands to that transmitting unit;

FIG. 6 is a plan view of an alternative embodiment of the patient unit of FIG. 2 having a graphic display;

FIG. 7 is a schematic cross-sectional representation of the graphic display employing an LED backlighting system with an LCD panel;

FIG. 8 is a perspective view of a shield container for the in-bore patient unit of FIG. 6 providing eddy-current reduction; and

FIG. 9 is a partial plan view of a patient showing a harness system for holding the patient unit of FIG. 2 to the patient in the bore for minimizing motion transmitting obstructions and lead entanglement.

### DETAILED DESCRIPTION OF THE PREFERRED EMBODIMENT

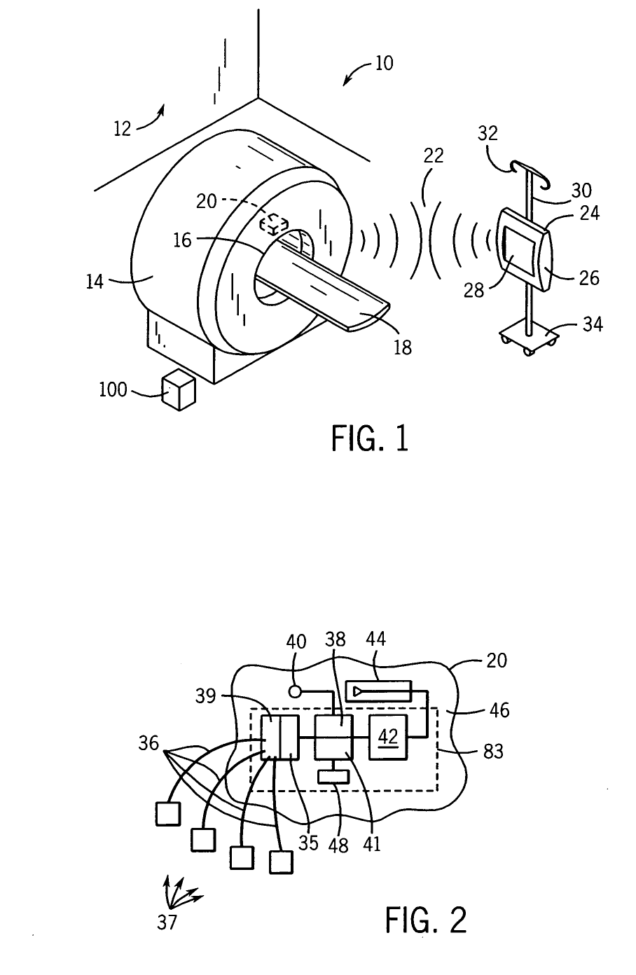

Referring now to FIG. 1, an MRI magnet room 10 containing an MRI magnet 14 may have shielded walls 12 blocking and reflecting radio waves. The MRI magnet 14 may have a central bore 16 for receiving a patient (not shown) supported on a patient table 18. As used henceforth, bore shall refer generally to the imaging volume of an MRI machine and should be considered to include the patient area between pole faces of open frame MRI systems.

During the MRI scan, the patient is held within the bore 16 and may be monitored via wireless patient unit 20 attached to the patient or patient table 18 and within the bore 16 during the scan. The patient unit 20 transmits via radio waves 22 physiological patient data and status data (as will be described) to processing unit 24 outside the bore 16 useable by personnel within the magnet room 10. The processing unit 24 typically will include controls 26 and a display 28 providing an interface for the operator, and may be usefully attached to an IV pole 30. The IV pole 30 may have hooks 32 for holding IV bags (not shown) and a rolling, weighted base 34 that may be freely positioned as appropriate without the concern for wires between the patient unit 20 and processing unit 24.

Referring now to FIG. 2, the patient unit 20 holds an interface circuit 35 for receiving physiological patient signals including, but not limited to, signals indicating: respiration, blood oxygen, blood pressure, pulse, and temperature, each from an appropriate sensor 37. Only ECG signals will be described henceforth for clarity.

When used to sense ECG signals, the interface circuit 35 may receive two or more ECG leads 36, being connected to, for example, the right arm, the right leg, the left arm and the left leg. The signals from these ECG leads 36 are connected to electrode amplifier and lead selector 39 which provides signals I, II and V, in a normal lead mode to be described below, or signals X, Y and Z in a vector lead mode (not shown), each attached to a corresponding electrode providing the sensor 37. The leads 36 may be high impedance leads so as to reduce the induction of eddy currents within those leads during the MRI process. The electrode amplifier and lead selector 39 provides the signals to an interface circuit 35 which controls signal offset and amplification, provides a gradient filter having variable filter settings to reduce interference from the MRI gradient fields, and converts the signals to digital words that may be transmitted to a contained processor 38. In a preferred embodiment, the ECG signals are sampled and digitized at a rate of 1,000 samples per second or faster so that they may be used for gating purposes. Other signals, such as those of blood oxygen may be sampled at a slower rate, for example, 250 samples per second.

The processor 38 communicates with flash memory 41 which may be used to buffer and store data from ECG leads 36 and which may have a stored program controlling the operation of the patient unit 20 as will be described below.

The processor 38 may communicate with an operator indicator 40, in this case a bi-colored LED, which may display operating information according to the following states:

| LED color | Meaning |
|---|---|
| Blinking Green | Good ECG Signals |
| Solid Green | No ECG Signal |
| Blinking Red | ECG, Poor |
| | Communication |
| Solid Red | No ECG, Poor |
| | Communication |

The operator indicator 40 has a lens which protrudes from a housing of the patient unit 20 so that it can be viewed by an operator sighting along the bore from a variety of attitudes. Importantly, the operator indicator 40 may be used during preparation of the patient outside of the bore, even in the absence of the processing unit 24 in the patient's hospital room.

The processor 38 of the patient unit 20 may also communicate with a transceiver 42. A suitable transceiver 42 provides multi-band Gaussian frequency shift keying (GFSK) in the 2.4 GHz ISM band and is capable of operating on battery power levels to produce powers of 0 dBm such as a type commercially available from Nordic Semiconductors of Norway under the trade name nRF24E1.

The transceiver 42 provides for transmission and reception of digital data packets holding samples of the ECG data with calculated error-correction codes over radio channels that may be selected by processor 38. Preferably the radio channels are selected to provide a substantial frequency difference between the channels to reduce the possibility of any interfering source of radio frequency from blocking both channels at the same time. The selection of channels 1 and 9 provide for an 8 MHz separation between channels.

The transceiver 42 connects to a microstrip antenna 44 which may be wholly contained within a housing 46 of the patient unit 20 outside of Faraday shield 83 to be described in more detail below. The housing 46, may for example be an insulating plastic material or other material. A battery 48 having no ferromagnetic terminal or other components, such as a polymer battery, is used to provide power to each of the interface circuit 35, processor 38, transceiver 42 and operator indicator 40, all held within the Faraday shield 83.

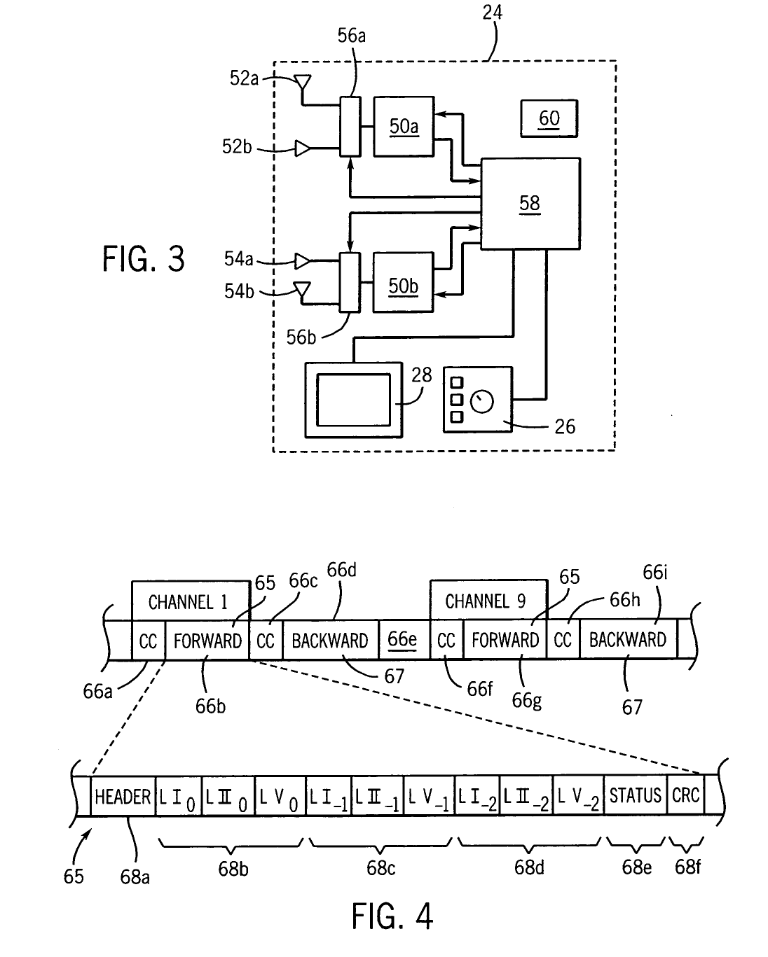

Referring now to FIG. 3, the processing unit 24 contains two transceivers 50a and 50b compatible with transceiver 42, and each switching between one of at least two channels depending on the frequency of transmission by the transceiver 42. Each of the transceivers 50 and 50b are connected to two antennas: antennas 52a and 52b for transceiver 50a, and antennas 54a and 54b for transceiver 50b, via a solid-state antenna switches 56a and 56b, respectively. A controller 58 receives data from and provides data to each of transceivers 50a and 50b for communication with the patient unit 20. The controller 58 also provides signals to the switches 56a and 56b to control which antennas are connected to transceiver 50a and 50b.

Antennas 52 and 54 are both spatially diverse and have different polarizations. Ideally, antennas 52a and 54a are vertically polarized and antennas 52b and 54b are horizontally polarized. Further, the antennas 52 and 54 are spaced from each other by approximately an odd multiple of a quarter wavelength of the frequencies of transmission by the patient unit 20 representing an expected separation of nodal points. This spacing will be an odd multiple of approximately 3 cm in the 2.4 GHz ISM frequency band.

With these diverse antennas 52a, 52b, 54a, and 54b, drop-off or adverse polarization of the waves at the processing unit 24, may be accommodated by switching of the antennas 52 and 54. Generally, this switching may be triggered when the signal from a given transceiver 50a or 50b is indicated to be corrupted by the error-correction code attached to data packets received by the given transceiver 50a or 50b as detected by program executed by the controller 58. Alternatively, the signal quality, for example, the signal strength or the length of time that the signal has been above a predetermined threshold, may be used to trigger the switching to the better of the two antennas 52 and 54.

The controller 58 communicates with a memory 60 such as may be used to store data and a program controlling operation of the processing unit 24. The controller 58 may also communicates with the display 28 that may display the physiological data collected by the patient unit 20 and user controls 26 that allow programming of that processing unit 24 and control of the display 28 according to methods well-known in the art.

Referring now to FIGS. 2 and 4, during operation, the processor 38 of the patient unit 20 executes a stored program in memory 60 to collect data from ECG leads 36 and to transmit it in time-diverse forward data packets 65 over multiple time frames 66. During a first time frame 66a, the processor 38 may switch the frequency of transmission of the transceiver 42 and provide a settling period of approximately 220 microseconds. As will be described, the frequency need not be changed at this time, but allowance is made for that change.

At time frame 66b, forward data packet 65, being physiological data from the patient, is transmitted from patient unit 20 to processing unit 24. This forward data packet will include a header 68a which generally provides data needed to synchronize communication between transceivers 42 and 50a and 50b, and which identifies the particular data packet as a forward data packet 65 and identifies the type of physiological data, e.g.: ECG, SPO₂, etc.

Following the header 68a, data 68b may be transmitted providing current samples in 16-bit digital form for the ECG signals at the current sampling time (e.g., LI₀, LII₀, LV₀). This is followed by data 68c providing corresponding samples in 16 bit digital form for the ECG signals at the next earlier sampling time (e.g., LI₋₁, LII₋₁, LV₋₁) as buffered in the patient unit 20. This in turn is followed by data 68d providing corresponding samples in 16 bit digital form for the ECG signals at the next earlier sampling time before data 68d (e.g., LI₋₂, LII₋₂, LV₋₂) again as buffered in the patient unit 20. In the vector mode, the samples may be Xₙ, Yₙ, and Zₙ.

Thus, a rolling window of three successive sample periods (one new sample and the two previous samples for each lead) is provided for each forward data packet 65. This time diversity allows data to be transmitted even if two successive forward data packets 65 are corrupted by interference.

Status data 68e follows data 68c and provides non-physiological data from the patient unit 20 indicating generally the status of the patient unit 20 including, for the example of ECG data, measurements of lead impedance, device temperature, operating time, battery status, test information, information about the lead types selected, the gradient filter settings selected, and the next or last radio channel to be used to coordinate the transceivers 42 and 50a and 50b. The status data 68e may also include a sequence number allowing the detection of lost forward data packet 65. Different status data 68e is sent in each forward data packet 65 as indexed by all or a portion of the bits of the sequence number. This minimized the length of each forward data packets 65.

Finally status data 68eincludes an error detection code 68f, for example, a cyclic redundancy code of a type well known in the art, computed over the total forward data packet 65 of header 68a, data 68b, data 68c, data 68d, and status data 68e that allows detection of corruption of the data during its transmission process by the controller 58. Detection of a corrupted forward data packet 65 using this error detection code 68f causes the controller to first see if an uncorrupted packet is available form the other transceiver 50a or 50b, and second to see if an uncorrupted packet is available from the following two forward packets. The antenna of the transceiver 50a or 50b is in any event switched to see if reception can be improved. Alternatively, signal quality, as described above, may be used to select among packets.

Referring still to FIG. 4, the forward data packet 65 of time frame 66b is followed by another channel changing time frame 66c which allows changing of the channel, if necessary, which is followed by a backward data packet 67 of time frame 66d providing data from the processing unit 24 to the patient unit 20.

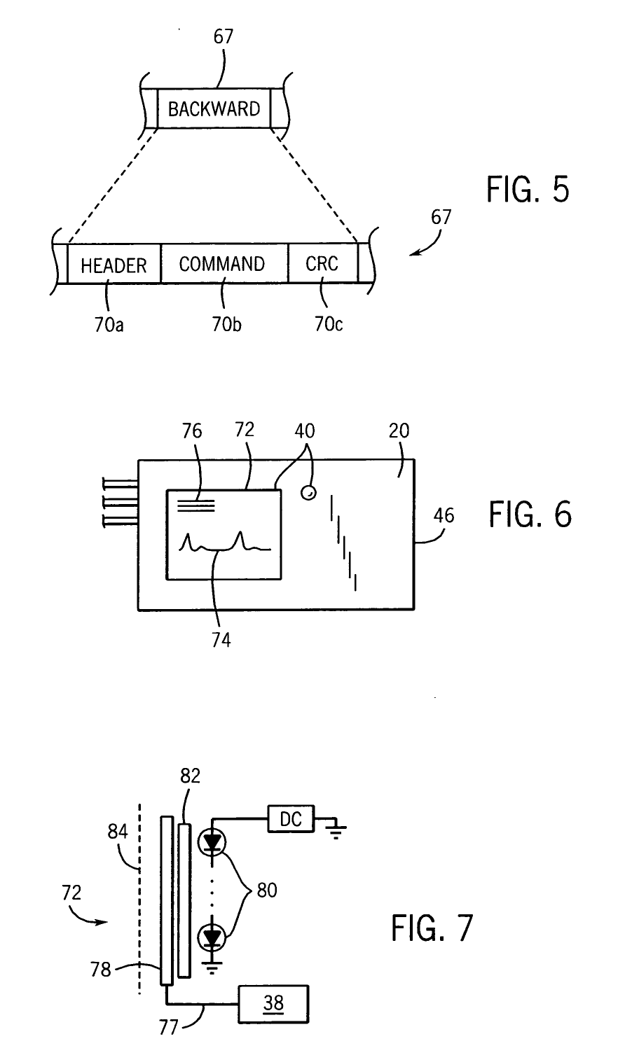

Referring now to FIG. 5, the backward data packet 67 may include a header frame 70a followed by command frame 70b and an error detection code 70c. The commands of the command frame 70b in this case may be instructions to the patient unit 20, for example, pulse the LED of the operator indicator 40 for testing or initiate a test of the hardware of the patient unit 20 according to diagnosis software contained therein, or to select the lead type of vector or normal described above, or to change the gradient filter parameters as implemented by the interface circuit 35, or to provide a calibration pulse, or to control the filling of flash memory on the patient unit 20 as may be desired.

Referring again to FIG. 4, an uncommitted time frame 66e may be provided for future use followed again by a channel change time frame 66f which typically will ensure that the radio channel used during the following forward data packet 65 of time frame 66g is different from the radio channel used in the previous forward data packet 65 of time frame 66b. This ensures frequency diversity in successive forward data packet 65 further reducing the possibility of loss of a given sample.

Referring now to FIG. 6, the present invention contemplates that the patient unit 20 may be used for setup of the patient without the need for processing unit 24, for example, in the patient's room before the patient is transported to the magnet room 10 or as a portable patient monitor that may be used for short periods of time in the patient room or during transportation of the patient and providing some of the features of the processing unit 24. For this purpose the patient unit 20 may include not only light for operator indicator 40, but graphic display 72 being similar to display 28 providing, for example, an output of physiological signal wave forms 74 and alphanumeric data 76.

Referring to FIG. 7, the display 72 to be suitable for use in the MRI environment, may comprise a liquid crystal panel 77 driven by processor 38 according to well known techniques but backlit by a series of solid state lamps, preferably white light-emitting diodes (LEDs) 80 communicating to the rear surface of the LCD panel 78 by a light pipe 82 instead of a common cold cathode fluorescent lamp. The LEDs 80 may be driven by a DC source to be unmodulated so as to reduce the possibility of creating radio frequency interference in the magnet bore caused by switching of the LEDs 80. The use of LEDs 80 also eliminates the high voltage interference that can occur from operation of cold cathode fluorescent tubes and the magnet components inherent in such tubes.

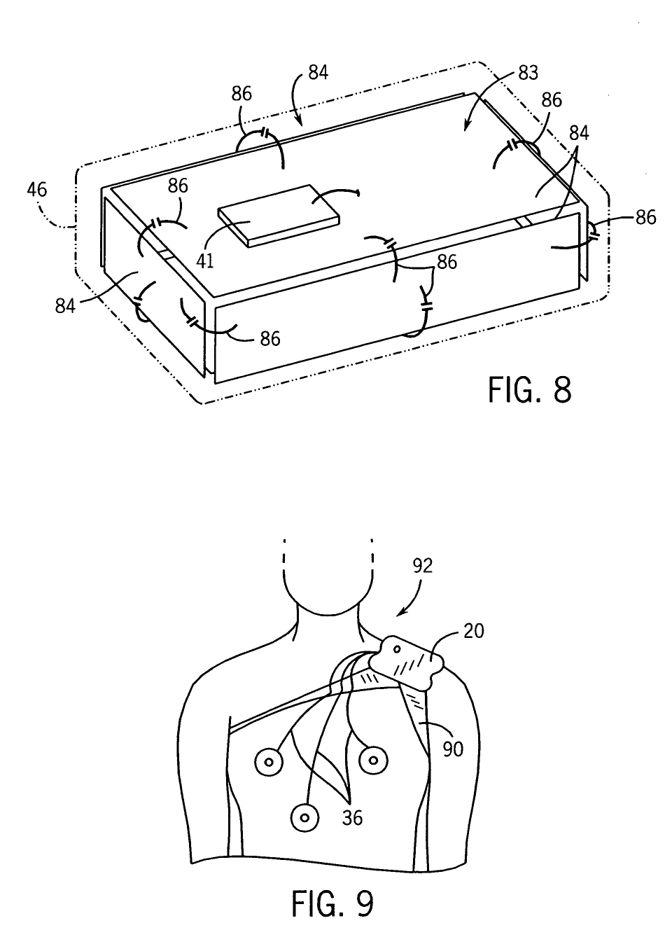

Referring now to FIG. 8, the circuitry of the patient unit 20 shown in FIG. 2, with the exception of the microstrip antenna 44, may be contained within a Faraday shield 83 held within the housing 46 and comprised of a box of conductive elements 84 formed of a mesh material, such as a screen or wire cloth. The microstrip antenna 44 may connect with the circuitry of the patient unit 20 with a conductor threaded through the mesh, through a waveguide, or a small aperture in the mesh, which blocks only free space radio frequency electromagnetic signals. The screen elements 84 may provide a mesh size smaller than the wavelength of the MRI gradient fields but ample to allow the display 72 to be viewed therethrough. Alternatively, the display 72 may be positioned outside of the Faraday shield 83. The light (preferably an LED) for the operator indicator 40 may protrude through the Faraday shield 83 to provide greater visibility to an operator outside the magnet bore.

The screen elements 84 providing radio frequency shielding for each face of the box forming the Faraday shield 83 may be insulated from each other with respect to direct currents, but yet joined by capacitors 86 at the corner edges of the box to allow the passage of a radio frequency current. The effect of these capacitors is to block the flow of lower frequency eddy currents induced by the magnetic gradients such as can vibrate the patient unit 20 when it is positioned on the patient. Alternatively, the capacitors 86 may be replaced with resistors (not shown) to dissipate the eddy currents through resistive heating.

Referring now to FIG. 9, the patient unit 20 may desirably be held by a harness 90 to the body, for example the shoulder of the patient 92, so as to be free from interference with the patient while maintaining a position conducive to transmission of wireless operator indicator 40. As positioned on the shoulder of the patient 92, the microstrip antenna 44 is removed from the patient 92 for line of sight transmission out of the bore and the LED operator indicator 40 is exposed for viewing outside the magnet bore. The harness may provide a guide for the ECG leads 36 reducing their entanglement and simplifying installation of the unit on the patient 92.

Referring now to FIG. 1, the present invention further contemplates that a gating unit 100 may be positioned in the magnet room 10 to receive signals both from the processing unit 24 and patient unit 20, and thereby to generate gating signals that may be used for gating the MRI machine. This gating unit may eavesdrop on the transmissions between the patient unit 20 and the processing unit 24 reducing the transmission overhead required of using these signals for gating.

It is specifically intended that the present invention not be limited to the embodiments and illustrations contained herein, but include modified forms of those embodiments including portions of the embodiments and combinations of elements of different embodiments as come within the scope of the following claims. For example, the diversity techniques as described herein may be applicable to optical and other wireless transmission methods. In the case of optical transmission, for example, different frequencies of light, modulation types, modulation frequencies, polarizations, orientations may be used to provide diversity.

We claim:

1. An electronic patient monitor for MRI imaging comprising:
(a) at least one sensor receiving a patient signal from the patient;
(b) a transmitter communicating with the sensor to transmit the patient signal to a remote receiving device; and;
(c) a shield housing surrounding at least a portion of the electronic patient monitor to:
(i) block free-space radio frequency signals and
(ii) suppress vibrations caused by gradient field induced eddy currents.

2. The electronic patient monitor of claim 1 wherein the transmitter provides wireless communication to the remote receiving device.

3. The electronic patient monitor of claim 2 wherein the transmitter is a radio transmitter having an antenna attached to an outside of the shield housing and communicating with the wireless transmitter through an aperture in the shield housing.

4. The electronic patient monitor of claim 3 further including a shell surrounding the shield housing.

5. The electronic patient monitor of claim 4 wherein the antenna is covered by the shell.

6. The electronic patient monitor of claim 3 wherein the antenna is a microstrip antenna.

7. The electronic patient monitor of claim 1 wherein the shield housing comprises separate sections joined by eddy-current-blocking elements selected from the group consisting of capacitors and resistors.

8. The electronic patient monitor of claim 7 wherein the shield housing is a substantially rectangular parallelepiped having each face electrically joined to an adjacent face by DC blocking elements.

9. The electronic patient monitor of claim 1 wherein the shield housing is a conductive screen.

10. The electronic patient monitor of claim 9 including a display visible though the conductive screen.

11. The electronic patient monitor of claim 10 wherein the display is an LCD panel.

12. The electronic patient monitor of claim 1 further including an LED visible outside of the shield housing through at least one aperture in the shield housing.

13. The electronic patient monitor of claim 1 including a mount adapted to hold the electronic patient monitor to the patient.

14. The electronic patient monitor of claim 13 wherein the mount is adapted to hold the electronic patient monitor with the antenna removed from the patient.

15. The electronic patient monitor of claim 13 further including an LED visible outside of the shield housing through at least one aperture in the shield housing wherein the mount is adapted to hold the electronic patient monitor with the LED visible by a person observing the patient.

16. The electronic patient monitor of claim 13 wherein the mount attaches the electronic patient monitor to a patient's shoulder.

17. The electronic patient monitor of claim 13 wherein the mount is a harness fitting around the patient's body

18. The electronic patient monitor of claim 13 wherein the mount is a harness fitting around the patient's shoulder.

19. The electronic patient monitor of claim 13 wherein the mount is a harness including supports for leads attaching the sensor to the patient.

20. The electronic patient monitor of claim 1 further including batteries contained in the electronic patient monitor to power the electronic patient monitor.

21. The electronic patient monitor of claim 20 wherein the batteries are polymer batteries.
